(19) United States
(12) Patent Application Publication
(10) Pub. No.: US 2011/0251541 A1
(43) Pub. Date: Oct. 13, 2011

(54) Polyurethane Foam Dressing Material with Excellent Adhesive Property and Method for Preparing the Same

(75) Inventors: Hyun Jung Kim, Gyeonggi-do (KR); Kab Keun Kim, Gyeonggi-do (KR); Seung Moon Lee, Gyeonggi-do (KR); Il Kyu Park, Gyeonggi-do (KR)

(73) Assignee: DONGSUNG BIOPOL CO., LTD, Kyunggi-Do (KR)

(21) Appl. No.: 12/875,366

(22) Filed: Sep. 3, 2010

(30) Foreign Application Priority Data
Apr. 12, 2010 (KR) 10-2010-0033260

(51) Int. Cl. A61L 15/26; B05D 3/00; A61F 13/02

(52) U.S. Cl. 602/46; 427/331

(57) ABSTRACT

Disclosed is a polyurethane foam dressing material with an excellent adhesive property, and a method for preparing the same, an object of the present invention is to provide an adhesive polyurethane foam dressing material and a method for preparing the same, in which the adhesive polyurethane foam dressing material has an excellent exudates adsorption degree and moisturizing rate available for applying the wounds with a lot of exudates, and a silicone/acryl adhesive applies on the wound contact side in order to have favorable for treating a wound, and easily remove without a pain.

### CROSS-REFERENCE(S) TO RELATED APPLICATIONS

The present invention claims priority of Korean patent application number 10-2010-0033260, filed on Apr. 12, 2010, which is incorporated herein by reference in its entirety.

### TECHNICAL FIELD

The present invention relates to a polyurethane foam dressing material with an excellent adhesive property, and a method for preparing the same, and more specifically to a wound dressing material that is composed of including a silicone adhesive, an acryl adhesive, and a reaction inhibitor for effectively coating the adhesion layer, and a method for preparing the same.

### BACKGROUND ART

A skin of the human body is a tissue covering the surface of the body for physically and chemically protecting the body from the outer world. At the same time, the skin of the human body is an integral organ to stay alive for keeping a biochemical function that is needed for systemic metabolism. When getting hurt on the skin, such as various external wounds, a cut, a burn, a bedsore, and the like, the skin lose its function, so that a patient feels a pain until completely healing the wounds, and even the patient life is being threatened when the skin has a widespread damage.

That is why people all put a lot of hard work in the project for healing the wounds until now in human history, so that there were known cures in various ways, and the cures have been now developed. A moist environment treatment is becoming the main cure of wounds since 1962, in which the treatment is able to maintain a cut in the wet state so that a formation of an epithetical cell is quickly increased as compared to a dry state. From various results of researches for the moist environment treatment, various closing moist environment dressing materials have rapidly been developed and introduced, and the treatment for the wounds have been variously developed and applied, in which the dressing materials are for maintaining a cut in the wet state.

Representative type of currently known moist environment dressing materials is a film type, a hydrocolloid type, a hydro gel type, polyurethane type, and the like, and specifically, the hydrocolloid type, the hydro gel type, a polyurethane type, and the like are being used.

The hydro gel type disclosed in U.S. Pat. No. 5,501,661 and U.S. Pat. No. 5,489,262 is a type applying the hydro gel on a polymer film layer without a penetration property, in which the polymer film layer prevents the dehydration and dry of the hydro gel, and the hydro gel layer promotes a wound healing state by adsorbing exudates by contacting on the surface of wound, and maintaining the wet state. However, it is not available on the wound with a lot of exudates due to a low water vapor permeability and water absorption rate, and when high absorption occurs; there is a problem that it is difficult to exchange a dressing material.

The hydrocolloid type disclosed in U.S. Pat. No. 5,503,847 and U.S. Pat. No. 5,830,932 is composed of a hydrocolloid layer that alleviates an impact from an adhesion composition layer and outer world, and absorbs the exudates, and a film layer for preventing the penetration of a bacteria and a foreign body. The dressing material of the hydrocolloid type forms a gel by absorbing a small amount of wound secretion, supplies a wet environment, prevents an organ disorder by maintaining weakly acidic pH for a long time, and supplies the environment for promoting a cell growth. However, there are problems that the hydrocolloid type is unsuitable for applying the wound with a lot of wound secretion because the water vapor permeability and an ability of an exudates adsorption are lacking, and it should be secondary removed because the gel can be leaved as a residue by sticking the gel on the surface of wound when exchanging or removing the gel.

Further, a hydrophilic polyurethane foam dressing material disclosed in U.S. Pat. No. 5,445,604 and U.S. Pat. No. 5,065,752 has a structure of three layers that are laminated with films on both of the polyurethane foam, and a layer film contacted to the surface of wound for preventing an embedment of a large pore of the contact layer of contact area on the surface of wound has a mechanical hole to be absorbed the exudates on the contact layer of the surface of wound. However, secondary adhesive and fixed film are needed for fixing on the surface of wound when using a dressing because there is no adhesive property on the adhesive surface of wound, and one or many people are needed for handling as occasion demands, and suffer from lack of person for handling.

U.S. Pat. No. 6,051,747 and U.S. Pat. No. 6,207,875 disclose an adhesive foam dressing material improving the problems as motioned above. The hydrophilic polyurethane foam dressing material also has a mechanical hole on its porous form to smoothly adsorb the exudates. Further, in order to easily attach on the surface of wound, a silicone gel is applied on the contact layer of the surface of wound to improve a handling line, but it is unfavorable to apply on the wounds with a lot of exudates due to the low of exudates absorption rate and moisturizing rate.

### DISCLOSURE

### Technical Problem

For this reason, the inventors of the present invention repeated the research and experiment in order to solve the above-mentioned problems, so that the inventiors suggest the present invention. An object of the present invention is to provide an adhesive polyurethane foam dressing material and a method for preparing the same, in which the adhesive polyurethane foamdressing material has an excellent exudates adsorption rate and moisturizing rate available for applying the wounds with a lot of exudates, and a silicone/acryl adhesive applies on the wound contact area in order to have favorable for treating a wound, and easily remove without a pain.

### Technical Solution

To achieve the above object, the present invention provides a polyurethane foam dressing material with an excellent adhesive property, comprising a contact layer for wound area having a sponge strucutre, and a protect layer of a film type,

wherein the contact layer and the protect layer are laminated, and an adhesion layer is formed forward the wound area of the contact layer, and

wherein the adhesion layer is composed of a silicone adhesive and an acryl adhesive, in which the acryl adhesive is mixed with the amount of 1-40 parts by weight based on 100 parts by weight of the silicone adhesive.

Further, in order to achieve the above-metioned object, the present invention provides a method for preparing a polyurethan foam dressing material with an excellent adhesive property, comprising forming a protect layer of a film type and an adhesion layer on polyurethan forming foam,

wherein the forming of the adhesion layer includes mixing 1˜40 parts by weight of the acryl adhesive based on 100 parts by weight of the silicone adhesive, coating the mixed solution on the polyurethane foaming foam to a constant thickness, and then curing.

Hereinafter, an exemplary embodiment of the present invention will be described in detail.

The polyurethane foam dressing material with an excellent adhesive property according to the present invention is an adhesive wound dressing material, in which the adhesion layer is composed of silicone adhesive, a water-soluble acryl adhesive, and a reaction inhibitor when necessary, and the foam layer is composed of the urethane foaming foam.

The foam layer may use generally known polyurethane foam, and more preferably a polyurethane foam with a sponge structure composed of a plurality of open cells and pores penetrating the cells. Specifically, a moisturizing rate of foam is about 300˜1,200%, a pore area is about 10˜35% of the whole area of cells, the average diameter of the open cells is about 50˜300 μm, the average diameter of the pores is about 5˜85 μm, the rate of the open cells is about 20˜70%, a density of the open cells is about 0.15˜0.45 g/cm³ but having nothing to do with the density, the adsorption rate of the open cells is about 400˜2,000 wt %, and it has nothing to do with the thickness of foam.

The silicone adhesive is used for the stable adhesion with the skin, and may use a polydimethylsilozane with vinyl groups and a hydrogen-terminated dimethyl siloxane, repectively, or the mixture of 1:0.1˜0.1:1 rates thereof. Specifically, a soft skin adhesive (SSA), such as Dow Corning, Wacker, and BLUESTAR's silicones can be used, and for example SSA from Dow Corning is MG 7-9700, MG 7-9800, MG 7-9850, MG 7-9900, and the like.

The acryl adhesive is used for a miscibility of the silicone adhesion layer and the polyurethane foaming foam layer, and preferably is mixed with the amount of 1˜40 parts by weight, more preferably 5˜30 parts by weight, and most preferably 10˜20 parts by weight based on the silicone 100 parts by weight. In the case that the acryl adhesive is less than 1 part by weight based on the silicone 100 parts by weight, there is no miscibility effect of the silicone and the polyurethane foam; on the contrary, in the case that the acryl adhesive exceeds 40 parts by weight based on the silicone 100 parts by weight, it is difficult to prepare a homogenized solution because there is no miscibility of the silicone and the acryl adhesive. Further, it is more preferable that its range is in the range of 10 to 20 parts by weight rather than in the range of less than 10 parts by weight or above 20 parts by weight, as disclosed in the following Examples 1 and 3, because the miscibility with the silicone is excellent as well as not separating of foam layer and silicone/water-soluble acryl adhesion layer.

One or copolymerized resin consisting of a methylacrylate, an ethylacrylate, an octylacrylate, an ethylhexylacrylate, a butylmethacrylate, an acrylic acid and a methacrylic acid may be used as the type of the water-soluble acryl adhesive, but it is not limited thereto.

The reaction inhibitor (curing retarder) may use a cyclohexanol, a methylcyclohexanol, a dimethylcyclohexanol, an ethnylcyclohexanol, and the like, respectively, or the mixture of 1:1.0˜0.1:1 rate.

The reaction inhibitor used is mixed with the amount of 0.1˜2 parts by weight, more preferably 0.5˜1 parts by weight based on 100 parts by weight of silicone. In the case that the content is added with less than 0.1 parts by weight, the inhibition effect of the curing reaction is not properly carried out; on the contrary, in the case that the content exceeds 2 parts by weight, the curing is not quickly carried out after coating, so that these cases are not preferable.

The silicone adhesive, the acryl adhesive, and the reaction inhibitor (curing retarder) if needed are mixed as the above-mentioned rates, and then used for forming the adhesion layer when preparing the dressing material.

At this time, the forming of the adhesion layer includes mixing with the silicone adhesive, the acryl adhesive, and the reaction inhibitor, thin film coating the prepared mixture on the polyurethane foaming foam in the constant thickness using a thin film apparatus, and then curing.

At this time, after the urethane foaming foam covers on the film applied with the silicone/water-soluble acryl mixed solution in the ranges of 1˜10 mm foam thickness and 0.1˜1.0 density, the curing is carried out to prepare the polyurethane foaming foam. In the case that the thickness of the foam is less than 1.0 mm, there is a problem that the adhesion is difficult when using a product; on the contrary, in the case that the thickness of the foam exceeds 10.0 mm, there is a problem that an elimination of the contact area easily occurs. Further, in the case that the density is less than 0.1, there is a problem that the absorption rate and moisturizing rate are low; on the contrary, in the case that the density exceeds 1.0, there is a problem that the hardness is increased, thereby increasing a pain when eliminating thereof.

The curing is preferably carried out at an oven set with a temperature of 90˜110° C. for 3˜6 minutes. In the case that the temperature of the curing is less than 90° C., the curing is not carried out; on the contrary, in the case that the temperature of the curing exceeds 110° C., it is difficult to expect further curing. Further in the case that the time of the curing is less than 3 minutes, the curing is not carried out; on the contrary, in the case that the time of the curing exceeds 6 minutes, it is difficult to expect further curing.

The thickness of the adhesion layer is preferably 20˜95 μm after curing. In the case that the thickness is less than 20 μm, there are problems that the separation from the foam layer easily occurs and it is difficult to prepare; in the case that the thickness exceeds 95 μm, the absorption rate is decreased due to the complete close of the foam layer.

### ADVANTAGEOUS EFFECTS

As described above, the polyurethane foam dressing material according to the present invention can be easily prepared and has an excellent exudates adsorption rate and moisturizing rate, can be easily applied to wounds due to further adhesive property, and can be removed without a pain.

### DESCRIPTION OF DRAWINGS

The above and other objects, features and advantages of the present invention will become apparent from the following description of preferred embodiments given in conjunction with the accompanying drawings, in which:

FIG. 1 is a scanning electron microscope photograph (×50 magnifications) of a cross-section of contact layer of a polyurethane foam dressing material according to an example 1 of the present invention; and

FIG. 2 is a scanning electron microscope photograph (×50 magnifications) of a cross-section of contact layer of a polyurethane foam dressing material according to comparative example 1.

### BEST MODE

The range of the present invention is not limited to the following Examples, because Examples according to the present invention can be modified in various other types.

### Example 1

Two-component silicone A and B solution was used for preparing the silicone adhesion layer and the mixed rate of A and B solution was 1:1. Dow Corning MG 7-9700 as the silicone was used.

For the miscibility of the silicone layer and the polyurethane foaming foam layer, two types of silicone/acryl mixed solutions were prepared to add 10 parts by weight and 20 parts by weight of the water-soluble acryl adhesive respectively based on 100 parts by weight of the silicone mixed solutions.

The mixed silicone/acryl solution were applied on the two-component film that is easily able to separate from the silicone/acryl adhesion layer to be 10, 20, 40, 50, 60, 80, 95, and 110 μm of thickness after curing. After the urethane foaming foam (Thickness: 0.2 mm, Density: 0.2 g/cm³) covered on the film applied with the silicone/acryl mixed solution, the curing was carried out at 100° C. for 5 minutes to prepare the adhesive wound dressing material containing the silicone adhesive (adhesive polyurethane dressing material).

For the prepared 14-species wound dressing materials, the physical properties were measured as the following methods. The results were shown in the following Table 1 in the case of 10 parts by weight of the acryl adhesive, and the results were shown in the following Table 2 in the case of 20 parts by weight of the acryl adhesive.

### Comparative Example 1

### Only Foam

The polyurethane foaming foam without an adhesive was used. For Comparative Example 1 in the followings Table 1 and Table 2, Mepilex that is now available in the market was used.

### Comparative Example 2

### When Preparing an Adhesion Layer with Only an Acryl Adhesive

Example 1 was repeated, except that an adhesion layer with a thickness of 40 μm was prepared with the water-soluble polyacrylic acid adhesive.

### Comparative Example 3

### When Preparing an Adhesion Layer with Only a Silicone Adhesive

Example 1 was repeated, except that an adhesion layer with a thickness of 40 μm was prepared with Dow Corning MG 7-9700 as a silicone adhesive.

[Evaluation of Physical Property]

Confirmation of Surface of Adhesive Wound Dressing Material

A microscopic surface analysis was carried out for confirming the adhesion layer that is able to adsorb exudates of the adhesive dressing materials according to Example 1, and Comparative Example 1, 2, and 3.

For the surface analysis, the adhesive wound dressing material was coated with a platinum ion, and then located on Scanning Electron Microscope to confirm each sample according to Example 1, and Comparative Example 1, 2, and 3.

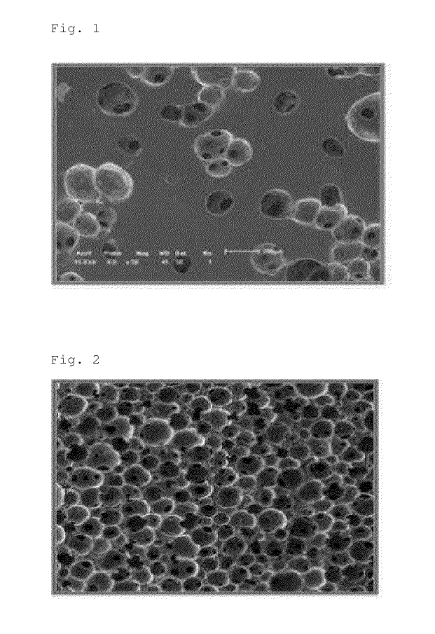

For reference, the Scanning Electron Microscope photograph (×50 magnifications) of a cross-section of contact layer of a polyurethane foam dressing material according to an example 1 of the present invention was shown in FIG. 1; and the Scanning Electron Microscope photograph (×50 magnifications) of a cross-section of contact layer of a polyurethane foam dressing material according to comparative example 1 was shown in FIG. 2.

As shown in FIGS. 1 and 2, the adhesive wound dressing material according to Example 1 of the present invention (FIG. 1) covers the porous urethane foam layer, and has a structure that is able to adsorb exudates due to the porous material.

On the contrary, there is no adhesion layer, so that the surface is porous according to only foam of Comparative Example 1(FIG. 2); According to Comparative Example 2 and 3 when using only the acryl adhesive and silicone adhesive, the structure can be confirmed that the adhesion layer and porous were coexisted as similar to the structure of the silicone/acryl mixed adhesive.

Comparative Test of Adhesion Strength of Adhesive Wound Dressing Material

The adhesion strength was performed by applying the samples prepared according to ASTM D3330 on Instron universal tester in order to confirm the adhesion strengths of adhesive dressing materials of Example 1, and Comparative Example 1, 2, and 3.

The ways for measuring includes locating and fixing the prepared specimens having 1.2 cm×15 cm size between grips, and then performing the adhesion strength test at 300 mm/minutes cross-head speed.

Initial Adsorption Speed of Adhesive Wound Dressing Material

PBS was dropped on the contact area of the dressing materials from 1 cm height and the adsorption speed of the dropped PBS was measured in order to measure an initial adsorption speed of the adhesive dressing materials according to Example 1, and Comparative Example 1, 2, and 3. The obtained results were shown in the following Table 1 and Table 2.

Measuring Adsorption Rate and Moisturizing Rate of Adhesive Wound Dressing Material

In order to measure the adsorption rate of the adhesive dressing material according to Example 1, and Comparative 1, 2, and 3, the wound dressing material having 3 cm×3 cm was taken, dried at 50° C. vacuum oven for 24 hours, and then weighted as the initial weight (a) after drying. The wound dressing material was impregnated in a distilled water of 25° C. for 24 hours, dried the surface of sample with a paper, and then weighted as the weight (b). And the adsorption rate was calculated as the following equation:

Adsorption Rate(%)=(b−a)/a×100 [Equation 1]

Further, in order to measure the moisturizing rate, the wound dressing material having 3 cm×3 cm was taken, dried at 50° C. vacuum oven for 24 hours, and then weighted as the initial weight (A) after drying. The wound dressing material was impregnated in a distilled water of 25° C. for 24 hours. After a roller with 3 kg weight was passed three times on the sample, the weight (C) was measured, and the moisturizing rate was calculated as the following equation:

Moisturizing Rate(%)=(C−A)/A×100 [Equation 2]

TABLE 1

| | Comparative | | | | | | | | | Comparative | Comparative |
|---|---|---|---|---|---|---|---|---|---|---|---|
| Physical | Example 1 | 10 parts by weight of acryl | | | | | | | | Example 2 | Example 3 |
| property | (foam only) | 10 μm | 20 μm | 40 μm | 50 μm | 60 μm | 80 μm | 95 μm | 110 μm | (acryl only) | (silicon only) |
| Ph | 7.31 | 6.88 | 6.93 | 6.93 | 6.88 | 6.89 | 6.91 | 6.87 | 6.92 | 6.92 | 6.92 |
| (DW pH 7.54) | | | | | | | | | | | |
| Adhesion strength | Not | 4 | 9 | 9 | 10 | 10 | 10 | 11 | 12 | Not | Separation of |
| (breaking) | detection | | | | | | | | | detection | silicon layer |
| (kgf/cm²) | | | | | | | | | | | and foam layer |
| Intitial | 3 | 15 | 15 | 15 | 16 | 16 | 17 | 17 | 55 | 4 | 16 |
| adsorption | | | | | | | | | | | |
| speed (sec) | | | | | | | | | | | |
| Moisturizing | 1.18 | 1.19 | 1.17 | 1.17 | 1.16 | 1.18 | 1.19 | 1.16 | 1.14 | 1.15 | 1.14 |
| rate | | | | | | | | | | | |
| Adsorption | 2.47 | 2.42 | 2.42 | 2.42 | 2.39 | 2.40 | 2.43 | 2.40 | 2.39 | 2.40 | 2.40 |
| rate | | | | | | | | | | | |

TABLE 2

| | Comparative | | | | | | | | | Comparative | Comparative |
|---|---|---|---|---|---|---|---|---|---|---|---|
| Physical | Example 1 | 20 parts by weight of acryl | | | | | | | | Example 2 | Example 3 |
| property | (foam only) | 10 μm | 20 μm | 40 μm | 50 μm | 60 μm | 80 μm | 95 μm | 110 μm | (acryl only) | (silicon only) |
| Ph | 7.31 | 6.77 | 6.75 | 6.75 | 6.66 | 6.94 | 6.83 | 6.79 | 6.78 | 6.88 | 6.72 |
| (DW pH 7.54) | | | | | | | | | | | |
| Adhesion strength | Not | 4 | 10 | 10 | 10 | 11 | 11 | 12 | 12 | Not | Separation of |
| (upon breaking) | detection | | | | | | | | | detection | silicon layer |
| (kgf/cm²) | | | | | | | | | | | and foam layer |
| Intitial | 3 | 15 | 15 | 15 | 16 | 16 | 17 | 17 | 55 | 4 | 16 |
| adsorption | | | | | | | | | | | |
| speed (sec) | | | | | | | | | | | |
| Moisturizing | 1.19 | 1.17 | 1.18 | 1.17 | 1.19 | 1.16 | 1.18 | 1.17 | 1.19 | 1.15 | 1.15 |
| rate | | | | | | | | | | | |
| Adsorption | 2.47 | 2.40 | 2.41 | 2.41 | 2.38 | 2.44 | 2.39 | 2.38 | 2.40 | 2.40 | 2.38 |
| rate | | | | | | | | | | | |

As shown in the above Table 1 and Table 2, the samples having various coating thicknesses and having different acryl contents were tested for the adhesion strength, it could be confirmed that the more the coating thickness is increased, the more the adhesion strength is slightly increased. However, the adhesion strength according to the acryl content was not changed. Further, in the case that the thickness of the adhesion layer is 10 μm, there are problems that the adhesion strength was low, and the separation of the adhesion layer and foam layer was easily carried out. In the case that the thickness of the adhesion layer exceeds 110 μm, there is a problem that the initial adsorption speed was low. In Comparative Example 2, the adhesion strength could not be measured because the acryl adhesive was absorbed to the foam, and in Comparative Example 3, there was a problem that the silicone layer and the foam layer were separated.

### Example 2

### Confirmation of Curing Time

Two-component silicone (Dow Corning 9700) A solution and B solution of 1:1 weight rate were prepared. In order to prevent a curing during preparing, the reaction inhibitor was added with 0.1, 0.3, 0.4, 0.5, 0.6, 0.7, and 3 parts by weight, respectively, based on the mixed solution. The solution mixed with the silicone A, the silicone B, and the reaction inhibitor was sufficiently agitated for 1 hour, and then the curing of the mixed solution was confirmed for 5 minutes and 100° C.

TABLE 3

| | A content of Reaction inhibitor (parts by weight) | | | | | | |
|---|---|---|---|---|---|---|---|
| Content | 0.1 | 0.3 | 0.4 | 0.5 | 0.6 | 0.7 | 3.0 |
| Curing (◯, X) | ◯ | ◯ | ◯ | ◯ | ◯ | ◯ | X |

As shown in the above Table 3, when 3 parts by weight not within the condition of the present invention, there was a problem that after curing for 5 minutes at 100° C., the curing was not carried out.

### Example 3

### Measurement of Physical Properties of Acryl Adhesive

100 parts by weight of two-component silicone (Dow Corning 9700) A solution and B solution with 1:1 weight rate was prepared, and then sufficiently agitated for 1 hour. The water-soluble polyacrylic acid adhesive was added with 5, 10, 20, and 30 parts by weight based on the silicone, and then sufficiently mixed for 30 minutes in order to improve the miscibility of the silicone and the urethane foaming foam in the mixed silicone solution. In order to remove a bubble of the prepared mixed solution, the mixed solution was sufficiently defoaming in a de-aerator for 1 hour to prepare the silicone/acryl mixed solution containing the acryl. The urethane foaming foam (Thickness: 2.0 mm, Density: 0.2 g/cm³) was prepared to prepare the adhesive wound dressing material, and the 50 μm of mixed silicone solution was applied on a release film that can be separated from the silicone adhesion layer.

After covering the urethane foaming foam (Thickness: 2.0 mm, Density: 0.2 g/cm³) on the film applied with the silicone/acryl mixed solution, the curing was carried out for 5 minutes at 100° C. to prepare the adhesive wound dressing material containing the silicone adhesive (the adhesive polyurethane dressing material).

Among the prepared dressing materials, when the acryl content is 5 parts by weight, there is a problem that the silicone/water-soluble acryl adhesion layer and the foam layer was separated, as compared to 10 parts by weight or 20 parts by weight. Therefore, it could be confirmed that the acryl content is more preferably used within the range of 10 to 20 parts by weight based on the 100 parts by weight of the silicone adhesive, rather than less than 10 parts by weight or at least 20 parts by weight.

### Example 4

### Test of Condition and Type of Applicable Foaming Foam

After covering the urethane foaming foam with the foam thicknesses of 0.5, 1.0, 2.0, 3.0, 4.0, 5.0, 10, and 20 mm, and foam densities of 0.01, 0.05, 0.1, 0.2, 0.3, and 1.0 g/cm³ on the film applied with the silicone/water-soluble acryl mixed solution as Example 1, the curing was carried out for 5 minutes at 100° C. to prepare the adhesive wound dressing materials.

The all kinds of the adhesive wound dressing materials could be easily prepared without the difference according to the foam thicknesses and densities if applying the silicone/water-soluble acryl adhesion layer on the foaming foam. Further, there were no problems for using thereof, and all kinds of products could be easily applied on the application areas.

1. A polyurethane foam dressing material with an excellent adhesive property, comprising a contact layer for wound area having a sponge strucutre, and a protect layer of a film type,
wherein the contact layer and the protect layer are laminated, and an adhesion layer is formed forward the wound area of the contact layer, and
wherein the adhesion layer is composed of a silicone adhesive and an acryl adhesive, in which the acryl adhesive is mixed with the amount of 1˜40 parts by weight based on 100 parts by weight of the silicone adhesive.

2. The polyurethane foam dressing material with an excellent adhesive property of claim 1, wherein the adhesion layer is composed of further including 0.1˜2 parts by weight of a reaction inhibitor based on 100 parts by weight of the silicone adhesive.

3. The polyurethane foam dressing material with an excellent adhesive property of claim 1, wherein the thickness of the adhesion layer is 20˜95 μm.

4. The polyurethane foam dressing material with an excellent adhesive property of claim 1, wherein the silicone adhesive is at least one selected from the group consisting of a polydimethyl siloxane with vinyl groups and a hydrogen-terminated dimethyl siloxane.

5. The polyurethane foam dressing material with an excellent adhesive property of claim 1, wherein the monomer of the acryl adhesive is one selected from the group consisting of one or copolymerized resin of a methylacrylate, an ethylacrylate, an octylacrylate, an ethylhexylacrylate, a butylmethacrylate, an acrylic acid and a methacrylic acid as a water-soluble acryl adhesive.

6. The polyurethane foam dressing material with an excellent adhesive property of claim 1, wherein the acryl adhesive is mixed with the amount of 10˜20 parts by weight based on 100 parts by weight of the silicone adhesive.

7. The polyurethane foam dressing material with an excellent adhesive property of claim 2, wherein the reaction inhibitor is at least one selected from the group consisting of a cyclohexanol, a methylcyclohexanol, a dimethylcyclohexanol, and an ethnylcyclohexanol.

8. A method for preparing a polyurethan foam dressing material with an excellent adhesive property, comprising forming a protect layer of a film type and an adhesion layer on polyurethan forming foam,
wherein the forming of the adhesion layer includes mixing 1˜40 parts by weight of the acryl adhesive based on 100 parts by weight of the silicone adhesive, coating the mixed solution on the polyurethane foaming foam to a constant thickness, and then curing.

9. The method for preparing the polyurethan foam dressing material with an excellent adhesive property of claim 8, wherein the adhesion layer is composed of further mixing 0.1˜2 parts by weight of a reaction inhibitor based on 100 parts by weight of the silicone adhesive.

10. The method for preparing the polyurethan foam dressing material with an excellent adhesive property of claim 8, wherein the constant thickness is to be 20˜95 μm after curing thereof.
